(19) United States
(12) Patent Application Publication
(10) Pub. No.: US 2004/0009986 A1
(43) Pub. Date: Jan. 15, 2004

(54) Triglyceride depressant composition

(75) Inventors: Tsuneki Ohsawa, Tokyo (JP); Ikuo Takagi, Matsudo-shi (JP); Ippei Shimizu, Tokyo (JP); Tatsuhito Kondo, Tokyo (JP); Masato Nakayama, Kitakatsushika-gun (JP); Yasuhiro Torizumi, Ryugasaki-shi (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: SANKYO COMPANY, LIMITED, Tokyo (JP)

(21) Appl. No.: 10/465,436

(22) Filed: Jun. 18, 2003

Related U.S. Application Data
(63) Continuation-in-part of application No. PCT/JP01/10912, filed on Dec. 12, 2001.

(30) Foreign Application Priority Data
Dec. 18, 2000 (JP) 2000-383051
Dec. 14, 2001 (JP) 2001-380783
Sep. 6, 2002 (JP) 2002-249431

(51) Int. Cl. A61K 31/498; A61K 31/519; A61K 31/444; A61K 31/375; A61K 31/366; A61K 31/355; A61K 31/225

(52) U.S. Cl. 514/250; 514/332; 514/458; 514/474; 514/460; 514/548

(57) ABSTRACT

A blood triglyceride depressant composition which comprises pravastatin and one or more members selected from the group consisting of (1) pantethine, (2) inositol hexanicotinate, (3) a combination drug containing a riboflavin compound, a tocopherol compound and an ascorbic acid compound, and (4) a combination drug containing a tocopherol compound and an ascorbic acid compound.

This is Continuation-in-Part Application of International Application No. PCT/JP01/10912 filed Dec. 12, 2001 which is incorporated herein by reference in its entirety.

### BACKGROUND OF THE INVENTION

The present invention relates to a triglyceride depressant composition that contains pravastatin in combination with one or more substances selected from the group consisting of (1) pantethine, (2) inositol hexanicotinate, (3) a combination drug that contains a riboflavin derivative, a tocopherol derivative and an ascorbic acid derivative, and (4) a combination drug that contains a tocopherol derivative and an ascorbic acid derivative.

The relationships between triglyceride levels in the blood and arteriosclerotic diseases are not fully understood, unlike the clearly established relationships between blood cholesterol levels and coronary arteriosclerotic diseases. However, dyslipidemia is becoming increasingly more recognized as a risk factor for arteriosclerotic diseases. In addition, it has become clear that hyperglyceridemia induces insulin resistance and significantly contributes to arteriosclerosis (References: eg., Modern Physician, Vol. 18 No.1, 1998, pp. 53-56, and pp. 69-71).

Pravastatin reduces total cholesterol levels in the blood by inhibiting HMG-CoA reductase activity. Furthermore, it is known that pravastatin reduces triglyceride levels in the blood.

In addition, pantethine, inositol hexanicotinate, a combination drug containing a riboflavin derivative, a tocopherol derivative and an ascorbic acid derivative, and a combination drug containing a tocopherol derivative and an ascorbic acid derivative are known to reduce triglyceride levels in the blood.

However, it remains unknown whether co-administration of pravastatin with one of pantethine, inositol hexanicotinate, a combination drug that contains a riboflavin derivative, a tocopherol derivative and an ascorbic acid derivative and a combination drug that contains a tocopherol derivative and an ascorbic acid derivative synergistically decreases triglyceride levels in the blood. In addition, it remains unknown whether co-administration of pravastatin and a combination drug that contains a tocopherol derivative and an ascorbic acid derivative synergistically decreases triglyceride levels in the blood.

### BRIEF SUMMARY OF THE INVENTION

The present invention is a drug composition that reduces triglyceride levels in the blood. The drug composition contains pravastatin and one or more substances selected from (1) pantethine, (2) inositol hexanicotinate, (3) a combination drug that contains a riboflavin derivative, a tocopherol derivative and an ascorbic acid derivative, and (4) a combination drug that contains a tocopherol derivative and an ascorbic acid derivative.

### DETAILED DESCRIPTION OF THE INVENTION

The present inventors investigated drug compositions that decrease triglyceride levels in the blood, and found that co-administration of pravastatin with 1 or more substances selected from (1) pantethine, (2) inositol hexanicotinate, (3) a combination drug that contains a riboflavin derivative, a tocopherol derivative and an ascorbic acid derivative, and (4) a combination drug that contains a tocopherol derivative and an ascorbic acid derivative decrease triglyceride levels in the blood and completed the present invention.

Among these substances, a blood glyceride depressant composition that contains pravastatin and pantethine, a blood glyceride depressant composition that contains pravastatin and inositol hexanicotinate, a blood glyceride depressant composition that contains pravastatin and a combination drug that contains a riboflavin derivative, a tocopherol derivative and an ascorbic acid derivative, or a blood glyceride depressant composition that contains pravastatin and a combination drug that contains a tocopherol derivative and an ascorbic acid derivative are preferred.

Pravastatin (chemical name: (+)-(3R, 5R)-3,5-dihydroxy-7-[(1S,2S,6S,8S,8aR)-6-hydroxy-2-methyl-8-[(S)-2-methylbutyryloxy]-1,2,6,7,8,8a-hexahydro-1 -naphthyl]heptanoic acid) is a compound represented by the following chemical structure and its salts (particularly the sodium salt). Methods of manufacturing pravastatin have been disclosed in Japanese Patent Kokai Application No. SHO 57-2240 and so forth. Since pravastatin is commercially available, it is easily acquired.

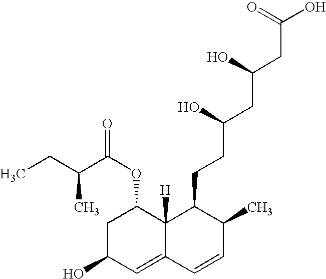

Inositol hexanicotinate is inositol whose 6 hydroxy groups are esterified with nicotinic acid.

The term ‘riboflavin derivative’ includes riboflavin and riboflavin esters such as riboflavin butyrate. Among these substances, riboflavin, riboflavin sodium phosphate, riboflavin butyrate, flavin-adenine dinucleotide, or flavin-adenine dinucleotide sodium salt are preferred.

The term ‘tocopherol derivative’ includes tocopherol (the racemate and its enantiomers) and esters of tocopherol such as tocopherol acetate (the racemate and its enantiomers). Among these substances, d-α-tocopherol succinate, dl-α-tocopherol succinate, dl-α-tocopherol calcium succinate, d-α-tocopherol acetate, dl-tocopherol acetate, d-α-tocopherol or dl-α-tocopherol are preferred. Furthermore, dl-α-tocopherol succinate or d-α-tocopherol acetate are more preferred, and particularly d-α-tocopherol acetate is the most preferred.

The term ‘ascorbic acid derivative’ includes ascorbic acid, ascorbates such as sodium ascorbate and ascorbic acid esters such as stearyl ascorbate. Among these substances, ascorbic acid, sodium ascorbate or calcium ascorbate are preferred and ascorbic acid is more preferred.

The term ‘blood triglyceride levels’ indicates total glyceride levels in the blood.

The term “depressant” in the expression “a blood triglyceride depressant composition” indicates that the levels are decreased by clinically significant amounts following administration of the composition.

The weight percent of pravastatin contained in solid preparations of the present invention of the blood triglyceride depressant composition is 0.01 to 5%, preferably 0.05 to 0.03%. The weight percent of pantethine in the solid preparations is typically 0.5 to 50%, preferably 1.0 to 25%. In addition, the weight percent of riboflavin derivative in the solid preparations is typically 0.002 to 40%, preferably 0.01 to 20%. Furthermore, the weight percent of ascorbic acid derivative is typically 0.05 to 50%, preferably 0.5 to 25%. The weight percent of tocopherol derivative is typically 0.002 to 40%, preferably 0.02 to 20%, and the weight percent of inositol hexanicotinate is typically 0.05 to 50%, preferably 0.5 to 25%.

The content of pravastatin contained in liquid and solution preparations of the blood triglyceride depressant compositions according to the present invention is typically 0.01 to 10 mg/mL, preferably 0.05 to 5 mg/mL; that of pantethine is typically 0.5 to 10 mg/mL, preferably 1 to 5 mg/mL; and that of riboflavin derivative is typically 0.05 to 5 mg/mL, preferably 0.1 to 3 mg/mL. In addition, the content of ascorbic acid derivative is typically 1 to 10 mg/mL, preferably 3 to 7 mg/mL. The content of tocopherol derivative is typically 0.5 to 5 mg/mL, preferably 1.5 to 3 mg/mL. The content of inositol hexanicotinate is typically 1 to 40 mg/mL, preferably 2 to 20 mg/mL.

Practical preparations of the drug compositions to reduce triglyceride levels in the blood are tablets, granules (involving powders), capsules, and liquids and solutions, etc., and are manufactured following addition of the required additive agents or materials, if necessary, according to conventional methods described in The Pharmacopeia of Japan.

In the preparations described above, additive agents that are conventionally used can be employed depending upon the preparation.

For instance, in the case of tablets, lactose and crystalline cellulose are used as diluents, magnesium aluminometasilicate, etc., are used as stabilizing agents, hydroxypropylcellulose, etc., are used as binders, and magnesium stearate is used as a lubricant.

In granules and capsules, lactose and purified sucrose, etc., are used as diluent, magnesium aluminometasilicate, etc., are used as stabilizing agent, cornstarch, etc., are used as an adsorbent, and hydroxypropylcellulose and polysorbate, etc., are used as a binder.

In liquids and solutions, D-sorbitol solution and honey, etc., are used as a sweetener, dl-malic acid, etc., are used as a flavoring agent, disodium dihydrogen ethylenediamine tetraacetate, etc., are used as a stabilizing agent, ethanol, etc., are used as a co-solvent, and polyoxyethylene hydrogenated castor oil stearate 60, etc., are used as a solubilizer.

In the preparations described above, a disintegrator such as crospovidone, etc.; an adsorbent such as calcium silicate, etc.; a coloring agent such as red ferric oxide and caramel, etc.; a pH modifier such as sodium benzoate, etc.; and a flavoring may be used if necessary.

When the composition in the present invention is administered, each component of the composition can be administered at the same time or individually at certain intervals.

The term “administration at the same time” described above has no particular limitation, provided that the components of the preparation are administered at roughly the same time. However, it is desirable to administer a single composition containing all components.

The term “administration of individual components at certain intervals” described above has no particular limitation, provided that each of the components of the preparation are individually administered at different times. In this case, one component is administered and the other components can then be administered within a defined time period.

In the case that 3 or more components in total are contained in the composition, the term “administration of these components at the same time or individually at different times” described above involves the following means of administration; all components are administered at the same time; all components are administered individually at different times; 2 or more components are administered at the same time and the remaining component(s) are administered at different times; 2 or more components are administered at different times and the remaining components are administered at the same time, and so on.

### TEST EXAMPLES

The present invention is described in more detail by way of the following examples. However, the present invention is not limited to these examples.

### Test Example 1

Tablets

(1) Composition

TABLE 1

| | Riboflavin/Ascorbic acid/ | |
|---|---|---|
| | Tocopherol | Pantethine |
| | 4 tablets | 4 tablets |
| | (1600 mg) | (1440 mg) |
| Pravastatin sodium | 20 mg | 20 mg |
| Riboflavin butyrate | 12 mg | — |
| Ascorbic acid | 500 mg | — |
| dl-α-Tocopherol succinate | 200 mg | — |
| Pantethine | — | 500 mg |
| Crystalline cellulose | 120 mg | 12 mg |
| Magnesium aluminometasilicate | 144 mg | — |
| Sucrose ester fatty acids | — | 140 mg |
| Hydroxypropylcellulose | 96 mg | 48 mg |
| Magnesium stearate | 24 mg | 24 mg |
| Crospovidone | 100 mg | 48 mg |
| Lactose | a | a |

a: appropriate quantity

TABLE 2

| | Inositol | Ascorbic acid + |
|---|---|---|
| | hexanicotinate | Tocopherol |
| | 4 tablets | 4 tablets |
| | (1,400 mg) | (1,400 mg) |
| Pravastatin sodium | 20 mg | 20 mg |
| Inositol hexanicotinate | 500 mg | — |
| Ascorbic acid | — | 500 mg |
| dl-α-Tocopherol succinate | — | 200 mg |
| Crystalline cellulose | 12 mg | 12 mg |
| Sucrose ester fatty acids | 140 mg | 140 mg |
| Hydroxypropylcellulose | 96 mg | 48 mg |
| Magnesium stearate | 24 mg | 24 mg |
| Crospovidone | 100 mg | 48 mg |
| Lactose | a | a |

a: appropriate quantity

(2) Manufacturing Methods

The amount of each component described above is weighed and prepared according to the methods described in the “General Rules for Preparations of Tablets” in The Pharmacopeia of Japan.

### Test Example 2

Granules

(1) Composition

TABLE 3

| | Riboflavin/Ascorbic acid/ | |
|---|---|---|
| | Tocopherol | Pantethine |
| | 4 packages | 4 packages |
| | (5.5 g) | (5.2 g) |
| Pravastatin sodium | 20 mg | 20 mg |
| Riboflavin butyrate | 12 mg | — |
| Ascorbic acid | 1.0 g | — |
| dl-α-Tocopherol succinate | 200 mg | — |
| Pantethine | — | 500 mg |
| Purified sucrose | 1.4 g | 1.6 g |
| Stevia extracts | — | 16 mg |
| Cornstarch | 1.2 g | 1.2 g |
| Polysorbate 80 | 80 mg | 48 mg |
| Magnesium aluminometasilicate | 144 mg | — |
| Magnesium stearate | 24 mg | 24 mg |
| Lactose | a | a |

a: appropriate quantity

TABLE 4

| | Inositol | Ascorbic acid + |
|---|---|---|
| | hexanicotinate | Tocopherol |
| | 4 packages | 4 packages |
| | (5 g) | (5 g) |
| Pravastatin sodium | 20 mg | 20 mg |
| Inositol hexanicotinate | 1000 mg | — |
| Ascorbic acid | — | 1000 mg |
| dl-α-Tocopherol succinate | — | 200 mg |
| Purified sucrose | 1400 mg | 1600 mg |
| Stevia extracts | 16 mg | 16 mg |
| Corn starch | 1200 mg | 1200 mg |
| Polysorbate 80 | 80 mg | 48 mg |
| Magnesium aluminometasilicate | 144 mg | 144 mg |
| Magnesium stearate | 24 mg | 24 mg |
| Lactose | a | a |

a: appropriate quantity

(2) Manufacturing Methods

The amount of each component described above is weighed and prepared according to the methods described in the “General Rules for Preparations of Granules” in The Pharmacopeia of Japan.

### Test Example 3

Capsules

(1) Components

TABLE 5

| | Riboflavin/Ascorbic acid/ | |
|---|---|---|
| | Tocopherol | Pantethine |
| | 8 capsules | 8 capsules |
| Pravastatin sodium | 20 mg | 20 mg |
| Riboflavin butyrate | 12 mg | — |
| Ascorbic acid | 500 mg | — |
| dl-α-Tocopherol succinate | 200 mg | — |
| Pantethine | — | 500 mg |
| Cornstarch | 960 mg | 960 mg |
| Polysorbate 80 | 80 mg | 48 mg |
| Magnesium aluminometasilicate | 144 mg | — |
| Magnesium stearate | 24 mg | 24 mg |
| Lactose | a | a |
| Subtotal | 2000 mg | 1940 mg |
| Capsule | 640 mg | 640 mg |
| Total | 2640 mg | 2580 mg |

a: appropriate quantity

TABLE 6

| | Inositol | Ascorbic acid + |
|---|---|---|
| | hexanicotinate | Tocopherol |
| | 8 capsules | 8 capsules |
| Pravastatin sodium | 20 mg | 20 mg |
| Inositol hexanicotinate | 500 mg | — |
| Ascorbic acid | — | 500 mg |
| dl-α-Tocopherol succinate | — | 200 mg |
| Corn starch | 960 mg | 960 mg |
| Polysorbate 80 | 80 mg | 48 mg |
| Magnesium aluminometasilicate | 144 mg | 144 mg |
| Magnesium stearate | 24 mg | 24 mg |
| Lactose | a | a |
| Subtotal | 2000 mg | 2000 mg |
| Capsule | 640 mg | 640 mg |
| Total | 2640 mg | 2640 mg |

a: appropriate quantity

(2) Manufacturing Methods

The amount of each component described above is weighed and prepared according to the methods described in the “General Rules for Preparations of Granules” in The Pharmacopeia of Japan, and hard capsules are prepared by filling the granules into capsules.

### Test Example 4

Liquids and Solutions

(1) Components

TABLE 7

| | Riboflavin/Ascorbic acid/ | |
|---|---|---|
| | Tocopherol | Pantethine |
| | 100 mL | 100 mL |
| Pravastatin sodium | 20 mg | 20 mg |
| Riboflavin sodium phosphate | 20 mg | — |
| Ascorbic acid | 500 mg | — |
| dl-α-Tocopherol succinate | 50 mg | — |
| Pantethine | — | 500 mg |
| D-Sorbitol solution (70%) | 4 g | 6 g |
| Honey | 7 g | 8 g |
| di-Malic acid | 200 mg | — |
| Disodium dihydrogen- | 20 mg | 20 mg |
| ethylenediamine tetrascetate | | |
| Ethanol | 2 mL | 2 mL |
| Polyoxyethylene hydrogenated- | 100 mg | 100 mg |
| castor oil stearate 60 | | |
| Sodium benzoate | 60 mg | 60 mg |
| Flavor | trace amount | trace amount |
| Distilled water | a | a |

a: appropriate quantity

TABLE 8

| | Inositol | Ascorbic acid + |
|---|---|---|
| | hexanicotinate | Tocopherol |
| | 8 capsules | 8 capsules |
| Pravastatin sodium | 20 mg | 20 mg |
| Inositol hexanicotinate | 500 mg | — |
| Ascorbic acid | — | 500 mg |
| dl-α-Tocopherol succinate | — | 50 mg |
| D-Sorbitol solution (70%) | 4 g | 6 g |
| Honey | 7 g | 8 g |
| dl-Malic acid | 200 mg | 200 mg |
| Disodium dihydrogen- | 20 mg | 20 mg |
| ethylenediamine tetraacetate | | |
| Ethanol | 2 mL | 2 mL |
| Polyoxyethylene hydrogenated- | 100 mg | 100 mg |
| castor oil stearate 60 | | |
| Sodium benzoate | 60 mg | 60 mg |
| Flavor | trace amount | trace amount |
| Distilled water | a | a |

a: appropriate quantity

(2) Manufacturing Methods

The amount of each component described above is weighed and prepared according to the methods described in the “General Rules for Preparations of Liquids and Solutions” in The Pharmacopeia of Japan.

Assay of Glycerol Levels in the Blood

(1) Test Compounds

Pravastatin with a purity of 99.4%, manufactured at Sankyo Co., Ltd., was used.

Pantethine, inositol hexanicotinate, riboflavin butyrate, d-α-tocopherol acetate, and ascorbic acid were purchased from Dai-ich Pharmaceutical Co., Ltd., SHIRATORI PHARMACEUTICAL CO., LTD., Mitsubishi-Tokyo Pharmaceutical Inc., Eisai Co., Ltd., and NIPPON ROCHE K. K., respectively.

(2) Test Animals

Beagle dogs aged 5 months were purchased from Covance Research Products Inc. and used after 1 month of quarantine and acclimatisation breeding.

(3) Preparation Forms for Administration, Methods of Preparing the Formulation, and Method of Stocking the Formulation

The calculated amounts of pravastatin or each component of the compositions based on the body weight of each dog were weighed and filled in a gelatin capsule (½ ounce) purchased from TORPAC Inc. Capsules filled with pravastatin were stocked in a refrigerator and those filled with combination drugs stocked at room temperature until use.

The combination drugs were filled in identical gelatin capsules.

(4) Route of Administration and Administration Period

Pravastatin or combination drug capsules were forcibly orally administered to each of the test animals once daily between 9:00 and 12:30. Animals were fasted for 2 or 3 hr prior to administration of the capsules.

The administration period was 11 successive days.

(5) Preparation of Test Samples and Procedures

Blood (10 mL) was collected from the superficial radial vein 14 and 7 days prior to capsule administration (2 and 1 weeks prior to administration) and 4, 8, and 12 days after administration of the capsule. Animals were fasted for approximately 18 hr prior to blood collection. Collected blood was placed in a test tube, left at room temperature for 0.5-1 hr, before being centrifuged (3,000 rpm for 10 min). The obtained serum was used for assays of blood triglyceride levels according to GK-GPO-POD methods. (Glycerokinase-Glycerophosphateoxidase-Peroxidase) methods, see: Kanai's Manual of Clinical Laboratory Medicine; 31^{st} Edition (September 1998), Kanehara & Co., Ltd.)

A full automatic analyzer (Monarch, Instrumentation Laboratory, Inc.) was used for assay of triglycerides.

Results

Lipid levels in the blood collected from dogs treated with either pantethine alone, inositol hexanicotinate alone, a composition which contains riboflavin butyrate, d-α-tocopherol acetate and ascorbic acid, a composition which contains d-α-tocopherol acetate and ascorbic acid, and a combination drug of pravastatin plus one of these substances, were converted to their relative ratios against their averaged pre-dosing levels (100) determined 2 and 1 weeks prior to the drug administration. The averaged value in each group was obtained from 5 animals per group.

(Effects of Co-Administration of Pravastatin and Pantethine)

TABLE 9

| | | Blood Triglyceride Levels | | |
|---|---|---|---|---|
| | Dose | after administration | | |
| Test Substance | (mg/kg) | 4 days | 8 days | 12 days |
| Pravastatin alone | 2 | 108.9 | 104.0 | 91.1 |
| Pantethine alone | 300 | 104.4 | 103.9 | 96.6 |
| Pravastatin + | 2 | 85.46 | 84.4 | 74.6 |
| pantethine | 300 | | | |

(Effects of co-administration of pravastatin and inositol hexanicotinate)

TABLE 10

| | | Blood Triglyceride Levels | | |
|---|---|---|---|---|
| | Dose | after administration | | |
| Test Substance | (mg/kg) | 4 days | 8 days | 12 days |
| Pravastatin alone | 2 | 108.9 | 104.0 | 91.1 |
| Inositol hexanicotinate alone | 400 | 109.3 | 94.8 | 111.7 |
| Pravastatin + | 2 | 79.4 | 81.1 | 86.7 |
| inositol hexanicotinate | 400 | | | |

(Effects of co-administration of pravastatin with a combination drug that contains riboflavin butyrate, d-α-tocopherol acetate and ascorbic acid)

TABLE 11

| | | Blood Triglyceride Levels | | |
|---|---|---|---|---|
| | Dose | after administration | | |
| Test Substance | (mg/kg) | 4 days | 8 days | 12 days |
| Pravastatin alone | 2 | 108.9 | 104.0 | 91.1 |
| Riboflavin butyrate | 6 | 104.2 | 98.8 | 86.0 |
| + d-α-Tocopherol acetate | 150 | | | |
| + Ascorbic acid | 500 | | | |
| Pravastatin | 2 | 77.4 | 80.3 | 74.8 |
| + Riboflavin butyrate | 6 | | | |
| + d-α-Tocopherol acetate | 150 | | | |
| + Ascorbic acid | 500 | | | |

(Effects of Co-Administration of Pravastatin with a Combination Drug which Contains d-α-tocopherol Acetate, and Ascorbic Acid)

TABLE 12

| | | Blood Triglyceride Levels | | |
|---|---|---|---|---|
| | Dose | after administration | | |
| Test Substance | (mg/kg) | 4 days | 8 days | 12 days |
| Pravastatin alone | 2 | 108.9 | 104.0 | 91.1 |
| d-α-Tocopherol acetate | 10 | 104.2 | 104.8 | 104.1 |
| + Ascorbic acid | 50 | | | |
| Proavastatin | 2 | 86.9 | 88.1 | 88.1 |
| + d-α-Tocopherol acetate | 10 | | | |
| + Ascorbic acid | 50 | | | |

The present invention, drug compositions of pravastatin in combination with pantethine and so forth, exhibits excellent blood triglyceride-lowering effects and is useful as a blood triglyceride-lowering agent.

Although the dose of compounds used according to the invention may widely vary depending on the extent of diseases and age of patients, (e.g. human patients), the dose of one administration of pravastatin is normally within the range of from 0.01 mg/kg to 10 mg/kg, preferably from 0.01 mg/kg to 1 mg/kg, administered once or several times a day dependent on the extent of diseases.

The dose of one administration of pantethine is normally within the range of from 0.06 mg/kg to 120 mg/kg, preferably from 0.6 mg/kg to 12 mg/kg, administered once or several times a day dependent on the extent of diseases.

The dose of one administration of inositol hexanicotinate is normally within the range of from 0.16 mg/kg to 36 mg/kg, preferably from 1.6 mg/kg to 3.6 mg/kg, administered once or several times a day dependent on the extent of diseases.

The dose of one administration of riboflavin derivative is normally within the range of from 0.004 mg/kg to 24 mg/kg, preferably from 0.04 mg/kg to 2.4 mg/kg, administered once or several times a day dependent on the extent of diseases.

The dose of one administration of tocopherol derivative is normally within the range of from 0.02 mg/kg to 60 mg/kg, preferably from 0.2 mg/kg to 6.0 mg/kg, administered once or several times a day dependent on the extent of diseases.

The dose of one administration of ascorbic acid derivative is normally within the range of from 0.1 mg/kg to 400 mg/kg, preferably from 1 mg/kg to 40 mg/kg, administered once or several times a day dependent on the extent of diseases.

What is claimed is:

1. A pharmaceutical composition comprising pharmacologically active agents in amounts effective in lowering blood triglyceride levels together with a carrier or diluent therefor, wherein said pharmacologically active agents comprise pravastatin and one or more agents selected from the group consisting of (1) pantethine, (2) inositol hexanicotinate, (3) a combination drug containing a riboflavin derivative, a tocopherol derivative and an ascorbic acid derivative, and (4) a combination drug containing a tocopherol derivative and an ascorbic acid derivative.

2. A pharmaceutical composition of claim 1 in solid dosage form containing 0.01 to 5% pravastatin and one or more compounds selected from the following group in the indicated amounts (1) 0.5 to 50 wt. % pantethine, (2) 0.05 to 50 wt. % inositol hexanicotinate, (3) 0.002 to 40 wt. % riboflavin derivative, 0.002 to 40 wt. % tocopherol derivative and 0.05 to 50 wt. % ascorbic acid derivative in a combination drug and (4) 0.002 to 40 wt. % tocopherol derivative and 0.05 to 50 wt. % ascorbic acid derivative in a combination drug.

3. A pharmaceutical composition according to claim 2 wherein said pharmacologically active agents comprise pravastatin and pantethine.

4. A pharmaceutical composition according to claim 2 wherein said pharmacologically active agents comprise pravastatin and inositol hexanicotinate.

5. A pharmaceutical composition according to claim 2 wherein said pharmacologically active agents comprise pravastatin and the combination drug containing a riboflavin derivative, a tocopherol derivative and an ascorbic acid derivative.

6. A pharmaceutical composition according to claim 2 wherein said pharmacologically active agents comprise pravastatin and the combination drug containing a tocopherol derivative and an ascorbic acid derivative.

7. A pharmaceutical composition of claim 1 in liquid or solution dosage form containing 0.01 to 10 mg/ml pravastatin and one or more compounds selected from the following group in the indicated amounts (1) 0.5 to 10 mg/ml pantethine, (2) 1 to 40 mg/ml inositol hexanicotinate, (3) 0.05 to 5 mg/ml riboflavin derivative, 0.05 to 5 mg/ml tocopherol derivative and 1 to 10 mg/ml ascorbic acid derivative in a combination drug and (4) 0.5 to 5 mg/ml tocopherol derivative and 1 to 10 mg/ml ascorbic acid derivative in a combination drug.

8. A pharmaceutical composition according to claim 7 wherein said pharmacologically active agents comprise pravastatin and pantethine.

9. A pharmaceutical composition according to claim 7 wherein said pharmacologically active agents comprise pravastatin and inositol hexanicotinate.

10. A pharmaceutical composition according to claim 7 wherein said pharmacologically active agents comprise pravastatin and the combination drug containing a riboflavin derivative, a tocopherol derivative and an ascorbic acid derivative.

11. A pharmaceutical composition according to claim 7 wherein said pharmacologically active agents comprise pravastatin and the combination drug containing a tocopherol derivative and an ascorbic acid derivative.

12. A method of lowering blood triglyceride levels, said method comprising administering, in combination, to a warm-blooded animal in need thereof, in amounts effective in lowering said blood triglyceride levels, pravastatin and one or more agents selected from the group consisting of (1) pantethine, (2) inositol hexanicotinate, (3) a combination drug containing a riboflavin derivative, a tocopherol derivative and an ascorbic acid derivative, and (4) a combination drug containing a tocopherol derivative and an ascorbic acid derivative.

13. The method of claim 12, wherein said pravastatin and said one or more substances selected from the group consisting of (1) pantethine, (2) inositol hexanicotinate, (3) said combination drug containing said riboflavin derivative, said tocopherol derivative and said ascorbic acid derivative, and (4) said combination drug containing said tocopherol derivative and said ascorbic acid derivative, are administered in the form of a combination pharmaceutical composition.

14. The method of claim 12, wherein said pravastatin and said one or more substances selected from the group consisting of (1) pantethine, (2) inositol hexanicotinate, (3) said combination drug containing said riboflavin derivative, said tocopherol derivative and said ascorbic acid derivative, and (4) said combination drug containing said tocopherol derivative and said ascorbic acid derivative, are administered separately and simultaneously.

15. The method of claim 12, wherein said pravastatin and said one or more substances selected from the group consisting of (1) pantethine, (2) inositol hexanicotinate, (3) said combination drug containing said riboflavin derivative, said tocopherol derivative and said ascorbic acid derivative, and (4) said combination drug containing said tocopherol derivative and said ascorbic acid derivative, are administered separately and non-simultaneously.

16. The method of claim 12 comprising administering pravastatin and pantethine.

17. The method of claim 12, wherein said pravastatin and said pantethine are administered in the form of a combination pharmaceutical composition.

18. The method of claim 12, wherein said pravastatin and said pantethine are administered separately and simultaneously.

19. The method of claim 12, wherein said pravastatin and said pantethine are administered separately and non-simultaneously.

20. The method of claim 12, comprising administering pravastatin and inositol hexanicotinate.

21. The method of claim 20, wherein said pravastatin and said inositol hexanicotinate are administered in the form of a combination pharmaceutical composition.

22. The method of claim 20, wherein said pravastatin and said inositol hexanicotinate are administered separately and simultaneously.

23. The method of claim 20, wherein said pravastatin and said inositol hexanicotinate are administered separately and non-simultaneously.

24. The method of claim 12 comprising administering pravastatin and a combination drug containing a riboflavin derivative, a tocopherol derivative and an ascorbic acid derivative.

25. The method of claim 24, wherein said pravastatin and said combination drug containing said riboflavin derivative, said tocopherol derivative and said ascorbic acid derivative are administered in the form of a combination pharmaceutical composition.

26. The method of claim 24, wherein said pravastatin and said combination drug containing said riboflavin derivative, said tocopherol derivative and said ascorbic acid derivative are administered separately and simultaneously.

27. The method of claim 24, wherein said pravastatin and said combination drug containing said riboflavin derivative, said tocopherol derivative and said ascorbic acid derivative are administered separately and non-simultaneously.

28. The method of claim 12 comprising administering pravastatin and a combination drug containing a tocopherol derivative and an ascorbic acid derivative.

29. The method of claim 28, wherein said pravastatin and said combination drug containing said tocopherol derivative and said ascorbic acid derivative are administered in the form of a combination pharmaceutical composition.

30. The method of claim 28, wherein said pravastatin and said combination drug containing said tocopherol derivative and said ascorbic acid derivative are administered separately and simultaneously.

31. The method of claim 28, wherein said pravastatin and said combination drug containing said tocopherol derivative and said ascorbic acid derivative are administered separately and non-simultaneously.

32. The method of claim 12, wherein said warm-blooded animal is a human.
